(19) United States
(12) Patent Application Publication
(10) Pub. No.: US 2007/0191956 A1
(43) Pub. Date: Aug. 16, 2007

(54) Radially extended support member for spinal nucleus implants and methods of use

(75) Inventors: Ann Prewett, Bloomfield Hills, MI (US); Gerald Gontarz, Spotswood, NJ (US)

Correspondence Address: CARTER, DELUCA, FARRELL & SCHMIDT, LLP, 445 BROAD HOLLOW ROAD, SUITE 225, MELVILLE, NY, 11747, US

(73) Assignee: Replication Medical, Inc.

(21) Appl. No.: 11/657,157

(22) Filed: Jan. 24, 2007

Related U.S. Application Data
(60) Provisional application No. 60/772,504, filed on Feb. 10, 2006.

(51) Int. Cl. A61F 2/44; A61F 2/46

(52) U.S. Cl. 623/17.16; 623/908; 264/241; 264/259

(57) ABSTRACT

A spinal nucleus implant is provided which includes an implant body and an interiorly embedded support member which extends out from the implant body. In one embodiment, the support member is fabric selected from the group consisting of mesh, woven fabric and nonwoven fabric. In one embodiment, the support member includes at least one portion which is located outside of the body, said portion adapted to engage one or more guides for orienting the implant. In one embodiment, the implant is capable of expanding from a compact, substantially dehydrated configuration to an expanded hydrated configuration. A method of manufacturing a spinal nucleus implant is provided which includes coagulating a liquid polymer such that at least a portion of said support member extends beyond the perimeter of the polymer to form a spinal nucleus implant having an interiorly disposed support member which extends out of the polymer. A method of implanting such a spinal nucleus implant is provided.

### CROSS REFERENCE TO RELATED APPLICATIONS

The present application claims the benefit and priority of provisional application Ser. No. 60/772,504 filed on Feb. 10, 2006 and titled RADIALLY EXTENDED SUPPORT MEMBER FOR SPINAL NUCLEUS IMPLANTS AND METHODS OF USE. The entire contents of Ser. No. 60/772,504 are hereby incorporated in its entirety herein.

### BACKGROUND

Spinal nucleus implants are known. For example, U.S. Pat. Nos. 5,562,736 and 5,674,295 disclose an implant having a constraining jacket surrounding a hydrogel core. As described therein, a hydrogel material is dehydrated, resulting in an undersized substantially cylindrical gel capsule which is then inserted into the constraining jacket which is then closed to prevent the hydrogel from escaping the confines of the jacket. The implant is rehydrated and conditioned by a series of compressive loads which renders the nucleus body to a partially flattened or oval shape. The implant is then inserted into a retaining tube to maintain the oval shape up until implantation. Alternative embodiments include an outer skin formed by ion implantation which causes outer layer polymerization and functions as the constraining jacket. U.S. Pat. No. 6,022,376 describes an implant made from an amorphous hydrogel polymer core surrounded by a constraining jacket. In one embodiment, the amorphous polymer is poured into one end of the constraining jacket in an unhydrated state, and the jacket then closed. The implant is then massaged to flatten and narrow the implant in preparation for implantation. Alternatively, the amorphous polymer may be injected into the constraining jacket. In one embodiment, an empty constraining jacket is implanted into the disc space and the amorphous polymer is then injected into the constraining jacket. In one embodiment, the amorphous polymer is shaped into a plurality of “microchips” which have been manufactured to have a certain shape. U.S. Pat. No. 6,132,465 is directed to a nucleus implant having a hydrogel core in a constraining jacket. The hydrogel core is inserted into the constraining jacket in a wedge-shaped dehydrated state and then implanted into the nucleus cavity. A final dehydration step is described where the hydrogel core can be forced into certain shapes, i.e., it can be “entirely flat”. U.S. Pat. No. 6,602,291 describes a prosthetic spinal disc nucleus which is made with a hydrogel core having a first shape in the hydrated state. It is then placed in a constraining jacket and reshaped to have a second shape in the dehydrated state. The core is configured to transition from the second shape to the first shape on hydration. The second shape may include an elongated shape defined by a leading end, the hydrogel core tapering from the central portion to the leading end, to facilitate insertion through an opening in the annulus. An inherent shape memory attribute is said to be obtained by pouring a hydrogel material, suspended in a solvent into a mold having a shape corresponding to the desired hydrated shape. After a solvent exchange process, the hydrogel core is dehydrated in an oven and inserted into a constraining jacket. The implant is then rehydrated and subjected to conditioning steps by exposure to at least three compressive loads. The implant is then reshaped and dehydrated, i.e., it is placed into a mold having a streamlined shape and then placed in an oven to expedite dehydration of the hydrogel core, which causes the implant to have a streamlined shape. The implant may be compressed while dehydrating. The implant is then maintained in the dehydrated shape prior to implantation. U.S. Pat. No. 6,533,817 is directed to a packaged, partially hydrated prosthetic disc nucleus which includes a prosthetic disc nucleus and a retainer. Upon contact with a hydration liquid, the retainer is said to be configured to allow the hydrogel core to hydrate from the dehydrated state but prevents the core from hydrating to the final hydrated state, i.e., the prosthetic disc nucleus is constrained by the retainer to a partially hydrated state. As described therein, a hydrogel core is formed and placed within a constraining jacket. The prosthetic disc nucleus is then dehydrated, preferably under compression within a compression mold and the entire assembly is placed in an oven. As the core dehydrates the compression mold forces the nucleus to a desired dehydrated shape in the dehydrated state. The dehydrated disc nucleus, in the dehydrated state is then placed in the retainer. The packaged disc nucleus can then be exposed to a hydration liquid where it transitions to the partially hydrated state. Once removed from the retainer, the disc nucleus, in the partially hydrated state is implanted into the disc space. U.S. Pat. No. 5,047,055 is directed to a hydrogel intervertebral disc nucleus. As described therein, a prosthetic nucleus for a disc is composed of a hydrogel material. The nucleus is made by mixing polyvinyl alcohol with a solvent heating the mixture and then poured or injected into a mold. The shaped hydrogel can be dehydrated for implantation. Other hydrogel materials are also described which can be shaped by cast molding or lathe cutting. The volume of the nucleus is said to reduce by about 80% when dehydrated and that the rigidity of the dehydrated nucleus will help the surgeons to manipulate the nucleus during an operation. U.S. Pat. No. 5,534,028 is directed to a hydrogel intervertebral disc nucleus with diminished lateral bulging and describes certain hydrogel treatment procedures which are similar to those disclosed in U.S. Pat. No. 5,047,055, e.g., see the implantation discussion at column 11, lines 25-40.

Surgical procedures for replacing or augmenting damaged or diseased nucleus pulposus involve anterior approaches or posterior approaches to the spinal column. The posterior approach (from the back of the patient) encounters the spinous process, superior articular process, and the inferior articular process to allow insertion of the disc replacement material into the intervertebral space, i.e., the bony sheath lies directly in front of each vertebral disc. The anterior approach to the spinal column is complicated by the internal organs that must be bypassed or circumvented to access the vertebrae. Thus, surgery is typically complicated and time consuming. An posterior-lateral aspect approach is the least invasive of these methods but provides limited and oblique access to the disc and its interior.

A potential shortcoming of artificial disc replacements is the propensity for extrusion of the implant through the annulus. The nucleus pulposus is held in place by the annulus in vivo. However, the annulus must be compromised in order to gain access to the diseased or damaged disc space. The resulting annular defect provides a path of least resistance through which a nucleus replacement or augmenter may travel under extremes of load and/or motion. In the case of implants which are made from a soft material, e.g., a hydrogel from polyvinyl alcohol, the propensity for extrusion through creep or flow is higher as the material gets softer. The likelihood of extrusion also increases with increased load.

The likelihood of extrusion occurring may further be increased by a poor implant cross-section to annular incision size ratio. The higher this ratio, the less likely it is that the implant will extrude. For example, if a 5 mm ø implant is placed into the disc space through a 5 mm ø incision the implant cross-section to annular incision ratio is 1.0 and extrusion is highly likely. It is therefore advantageous to keep this ratio as high as possible by reducing the incision size. This can be facilitated by decreasing the cross section of the implant which must pass through the annulus. In designing implants to be used with minimally invasive techniques, the cross-sectional area of the implant should be as small as possible. Although some of the above-described implants are dehydrated and shaped in some manner, none of them are dehydrated and reshaped so as to force the implant to assume an implantation-friendly shape substantially different from the final, hydrated implanted shape. Thus, the implant's original footprint may be maintained in the form of a wafer, which may have an aspect which is decreased along one axis, but not the other. Alternatively, isotropic shrinkage from dehydration may be effected which does not alter the topography of the implant. In the case of simple dehydration, the cross-sectional area is equal to the hydrated cross-sectional area divided by the expansion ratio.

Another method of optimizing the implant cross section for minimally invasive surgery is partial hydration of a hydrogel material which allows for manipulation of the implant by the surgeon with or without specialized tools designed for this purpose. There are a number of potential drawbacks to partial hydration or plastification such as incompatibility of the plasticizer used with the sterilization method, difficulty of retaining the required amount of plasticizer within the package over extended periods and the possibility of creep occurring during storage.

Accordingly there is a need to reduce the possibility that a spinal nucleus implant will extrude from the disc space through the annulus. Various methods have been proposed including physical barriers which span an annular defect. See, e.g., U.S. Pat. No. 6,883,520. Additional extrusion resistance may be obtained by mechanical attachment of the implant to the annulus by sutures, staples, clips and other fasteners. Such attachment methods may be problematic in the case of viscoelastic implants such as high water content hydrogels where the hydrogel matrix does not provide much resistance to tearing out of the fastener from the implant.

The present invention addresses at least these problems by providing a spinal nucleus implant which contains, inter alia, a novel interiorly embedded support member.

### SUMMARY

A spinal nucleus implant is provided which includes an implant body and an interiorly embedded support member which extends out from the implant body. In one embodiment, the body has an ellipsoid footprint. The interiorly embedded support member is preferably disposed within the implant body in substantially parallel orientation to the footprint and preferably extends beyond the body substantially parallel to the footprint. In one embodiment, the support member extends radially beyond and around the entire periphery of the body. In another embodiment, the support member extends beyond a defined portion(s) of the periphery of the body. In one embodiment, the support member is configured to extend and be folded over a portion of the surface area of the body. In one embodiment, the support member is configured to extend and be folded over a majority of the surface area if the body. In one embodiment, the support member is fabric selected from the group consisting of mesh, woven fabric and nonwoven fabric. The fabric may be made, e.g., from natural or synthetic polymers or metal fibers. In another embodiment, the support member is a foil made from metal or a polymer. In one embodiment, the body is made of at least two layers and the support member located between two layers. In one embodiment, the body is made of alternating substantially parallel layers wherein at least one of the layers contains the support member. In one embodiment, the support member is at least partially encapsulated by a polymeric coating. In one embodiment, the support member includes at least one portion which is located outside of the body, said portion adapted to engage a guide for orienting the implant. The guide may be selected from the group consisting of wire, ribbon or string. In one embodiment, a plurality of guides are attached to the support member. In one embodiment, the guide is releasably affixed to the support member. In another embodiment, the support member is adapted to promote ingrowth of tissue. In one embodiment, the support member incorporates a medicinal agent which promotes tissue growth. In one embodiment, the body is made of a hydrogel such as a polyacrylonitrile hydrogel. In one embodiment, the implant is capable of expanding from a compact, substantially dehydrated configuration to an expanded hydrated configuration.

A spinal nucleus implant is also provided which includes an implant body and an elongate flexible guide member affixed to the implant body. The guide member is preferably selected from the group consisting of wire, ribbon or string such as a suture. In one embodiment, the guide member is affixed to a support member which is embedded to the interior of the implant body. In one embodiment, the guide member is releasably affixed to the support member. In one embodiment, a plurality of guide members are attached to the support member. In one embodiment, the support member is fabric selected from the group consisting of mesh, woven fabric and nonwoven fabric. In another embodiment, the support member is a foil made from metal or a polymer. In one embodiment, the implant body is made of a hydrogel such as a polyacrylonitrile hydrogel. In one embodiment, the implant body incorporates layers, wherein certain layers have a different modulus of elasticity compared to other layers. In one embodiment, at least one of the layers includes a support member having a polymeric coating. In one embodiment, the implant is capable of expanding from a compact, substantially dehydrated configuration to an expanded hydrated configuration.

A method of manufacturing a spinal nucleus implant is provided which includes providing a liquid polymer, providing a mold for containing the polymer, providing a support member, positioning the support member relative to said mold such that liquid polymer can at least partially cover the support member, and coagulating the liquid polymer such that at least a portion of said support member extends beyond the perimeter of the polymer to form a spinal nucleus implant having an interiorly disposed support member which extends out of the polymer. In one embodiment, the mold includes a first ellipsoid ring portion for receiving liquid polymer and a second ellipsoid ring portion for disposing over the first ellipsoid ring portion and receiving liquid polymer, wherein positioning the support member relative to the mold involves filling the first ring with said liquid polymer, placing the support member over the first ring such that at least a portion of said support member extends beyond the perimeter of the first ring, positioning the second ring coaxially over the first ring and the support member to produce a substantially liquid-tight arrangement between the first and second rings, filling the second ring with liquid polymer, and coagulating the liquid polymer to form the spinal nucleus implant having an interiorly disposed support member which extends out of the polymer. In one embodiment, the method further includes providing a first additional ellipsoid ring mold, filling the first additional mold with liquid polymer, placing the implant having an interiorly disposed support member coaxially over the first additional ellipsoid ring mold and in contact with the liquid polymer, and coagulating the liquid polymer such that the polymer adheres to the implant having an interiorly disposed support member as it coagulates to form a spinal nucleus implant having a first polymeric layer containing the support member and a second polymeric layer, wherein the support member extends beyond the perimeter of the polymeric layers. In one embodiment, the first polymer layer containing the support member has a different modulus of elasticity than the second polymeric layer. In one embodiment, the method further includes providing a second additional ellipsoid ring mold, placing said second additional mold coaxially over the first polymer layer containing the support member, filling the mold with liquid polymer, and coagulating the liquid polymer such that the polymer adheres to the first polymer layer containing the support member as it coagulates, to form a three polymeric layer spinal nucleus implant wherein the support member extends beyond the perimeter of at least one of the polymeric layers. In one embodiment, the method further includes providing a second polymeric layer containing a support member, placing the second polymeric layer containing the support member coaxially over the second ellipsoid ring mold and in contact with the liquid polymer contained by the second ellipsoid ring mold, and coagulating the liquid polymer such that the polymer adheres to the second polymeric layer containing the support member as it coagulates, to form a four polymeric layer spinal nucleus implant. In one embodiment, the method further includes providing a third additional ellipsoid ring mold, placing said third additional mold coaxially over the second polymeric layer containing the support member, filling the third additional ellipsoid ring mold with liquid polymer, and coagulating the liquid polymer such that the polymer adheres to the second polymeric layer containing the support member as it coagulates, to form a five polymeric layer spinal nucleus implant. In one embodiment, the modulus of elasticity of the coagulated polymer of the polymeric layers having interiorly disposed support members is greater than the modulus of elasticity of the layers which do not have an interiorly disposed support member. In one embodiment, the liquid polymer is a hydrogel. In one embodiment, the hydrogel is a polyacrylonitrile hydrogel. In one embodiment, the support member is a fabric selected from the group consisting of woven, nonwoven and mesh. In another embodiment, the support member is a foil made from metal or a polymer. In one embodiment, at least one guide member is attached to the support member.

A method of implanting a spinal nucleus implant is provided which includes providing a spinal nucleus implant having a proximal portion and a distal portion, the distal portion having an elongated flexible guide member affixed thereto, the guide member having a proximal end and a distal end, the proximal end being affixed to the distal portion of the implant, providing a point of entry to the disc space between two vertebrae, inserting the implant into the disc space using the distal portion of the implant as the leading portion of the implant through the point of entry, manipulating the guide member to cause the implant to change position. In one embodiment, manipulating the guide member causes the implant to cant in arcuate fashion. In one embodiment, the distal portion of the implant follows an arc ranging from ˜45° to ˜100° relative to the proximal portion. The guide member may be selected from the group consisting of a string such as a suture, a wire and a ribbon. In one embodiment, the guide member is affixed to an interiorly embedded support member which extends out from the implant body, the guide member being affixed to a portion of the support member which extends out from the implant body. In one embodiment, the distal end of the guide remains outside the point of entry and manipulating the guide includes pulling on the guide member to pull the distal portion of the implant along the arc. In another embodiment, the method of implanting a spinal nucleus implant further includes providing a second point of entry to the disc space, using a grasping instrument to grasp the guide member from within the disc space, and using the grasping instrument to pull on the guide member and cause the implant to change position. In one embodiment, the change in position is a canting of the implant. In one embodiment, the proximal portion of the spinal implant has a second guide member attached thereto which may be used to manipulate the position of the implant. In one embodiment, the implant is fastened to a portion of the annulus using a fastener which fastens the support member to the annulus. In one embodiment, at least one guide member is at least partially radiopaque. In one embodiment, after the guide member has been manipulated to cause the implant to change position, at least a portion of the guide member is removed from the support member. In one embodiment, the at least a portion of the guide member is removed from the support member by cutting a portion of the guide member.

### BRIEF DESCRIPTION OF THE FIGURES

FIG. 1 is a top view of a spinal nucleus implant having an ellipsoid implant body and an interiorly embedded mesh support member spanning the entire body and extending out from opposite ends of the body.

FIG. 2 is a top view of a spinal nucleus implant having an ellipsoid implant body and an interiorly embedded mesh support member partially spanning the entire body and extending out from opposite ends of the body.

FIG. 3 is a top view of a spinal nucleus implant having an ellipsoid implant body and an interiorly embedded mesh support member spanning the entire body and extending out around the entire periphery of the body.

FIG. 4 is a top view of a spinal nucleus implant having an ellipsoid implant body and an interiorly embedded mesh support member partially spanning the entire body and extending out of a portion of the body.

FIG. 5 is a top view of a spinal nucleus implant having an ellipsoid implant body and an interiorly embedded foil support member spanning the entire body and extending out from opposite ends of the body.

FIG. 6 is a top view of a spinal nucleus implant having a kidney-shaped ellipsoid implant body and an interiorly embedded mesh support member spanning the entire body and extending out around the entire periphery of the body.

FIG. 7 is a side view of a spinal nucleus implant having a support member embedded interiorly and extending out beyond the perimeter of the implant body.

FIG. 8 is a side view of a multilayer spinal nucleus implant having five alternating substantially parallel layers, wherein the second and forth layers contain interiorly embedded support members. The support member of second layer extends out beyond the perimeter of the implant body.

FIG. 9 is a top view of a spinal nucleus implant having an ellipsoid implant body, an interiorly embedded mesh support member spanning the entire body and extending out from opposite ends of the body, and two guide members respectively affixed at opposite outwardly extending ends of the support member.

FIG. 10 is a schematic top view of an annulus surrounding a disc space, wherein a dehydrated spinal nucleus implant is shown partially inserted through the annulus into the disc space. A guide member extends from the leading edge of the implant back through the annulus.

FIG. 11 is a schematic top view of the annulus surrounding a disc space from FIG. 10, wherein the dehydrated spinal nucleus implant is shown completely inserted through the annulus into the disc space. The guide member extends from the leading edge of the implant back through the annulus. The schematic depicts the result of a slight pull on the guide member which causes the leading edge of the implant to cant sideways.

FIG. 12 is a schematic top view of the annulus, disc space and implant shown in FIGS. 10 and 11, wherein the guide member has been further pulled to cause the implant to cant transverse to its position when first inserted.

FIG. 13 is a schematic top view of an annulus surrounding a disc space, wherein a dehydrated spinal nucleus implant is shown partially inserted through a first point of entry in the annulus into the disc space. A first guide member extends from the leading edge of the implant through a second point of entry in the annulus. A second guide member is attached to the trailing edge of the implant.

FIG. 14 is a schematic top view of the annulus surrounding the disc space shown in FIG. 13, wherein the dehydrated spinal nucleus implant is shown completely inserted through the annulus into the disc space. The first guide member extends from the leading edge of the implant through the second point of entry in the annulus. The second guide member extends from the trailing edge of the implant back through the first point of entry in the annulus. The schematic depicts the result of a slight pull on the first guide member which causes the leading edge of the implant to cant sideways.

FIG. 15 is a schematic top view of the annulus, disc space and implant shown in FIGS. 13 and 14, wherein the first guide member has been further pulled to cause the implant to cant perpendicular to its position when first inserted. The second guide member is used to stabilize the proximal portion of the implant.

FIG. 16 is a schematic top view of an annulus surrounding a disc space, wherein a dehydrated spinal nucleus implant is shown partially inserted through the annulus into the disc space. A guide member extends from the leading edge of the implant and is contained with the disc space.

FIG. 17A is a schematic top view of the annulus surrounding a disc space from FIG. 16, wherein the dehydrated spinal nucleus implant is shown completely inserted through the annulus into the disc space. The guide member extends from the leading edge of the implant and is contained with the disc space.

FIG. 17B is a schematic top view of the annulus surrounding a disc space from FIG. 16, wherein the dehydrated spinal nucleus implant is still partially inserted through the annulus into the disc space. The guide member extends from the leading edge of the implant through the second point of entry in the annulus.

FIG. 18 is a schematic top view of the annulus surrounding the disc space shown in either FIG. 17A or 17B, wherein the dehydrated spinal nucleus implant is shown completely inserted through the annulus into the disc space. The guide member extends through a second point of entry. The schematic depicts the result of a slight pull on the guide member which causes the leading edge of the implant to cant sideways.

FIG. 19 is a schematic top view of the annulus, disc space and implant shown in FIGS. 16 through 18, wherein the guide member has been further pulled to cause the implant to cant perpendicular to its position when first inserted.

### DETAILED DESCRIPTION

A spinal nucleus implant (“SNI”) according to the present disclosure is uniquely suited for implantation into the disc space of a diseased or damaged intervertebral disc by virtue of a novel interiorly embedded support member which extends beyond the perimeter of the body of the implant. The support member is anchored in the body of the implant and provides reinforcement to the body of the implant which increases structural integrity, creep resistance and assists in preventing radial bulging of the implant under load bearing conditions. In addition, the portion of the support member which extends beyond the body of the implant provides an advantageous modality for guiding the implant into the disc space during implantation, anchoring the implant within the disc space, and/or providing a substrate for ingrowth of natural tissue, e.g., fibrous collagen, thus providing an additional anchoring mechanism for the implant.

A support member according to the present disclosure is suitable for use as a reinforcing element in any suitable polymeric-based SNI which can be formed from a liquid polymer. It is also suitable for use in any SNI (natural or synthetic) that is made from layers which are adhered to each other. The support member occupies at least a portion of the interior of the implant. The support member is preferably in the form of a fabric or a foil, but may also be a series of individual fibers or ribbons which are arranged in parallel or non-parallel fashion. The fabric may be woven or non-woven and may be in the form of a mesh. The size of interstices in the mesh is not deemed critical and it is contemplated that various mesh sizes are suitable. A fabric support member may be made of a polymeric material which is natural, e.g., cotton, or synthetic, e.g., polyester, polyamide, or other materials such as metal fiber, fiber glass, and carbon fiber. Methods of making fabric from these materials and others are well-known to those skilled in the art. Foils herein may also be made of metal or polymeric material and are well-known. Thus, the support member may be constructed from relatively durable materials including, but not limited to, metal foil, metal fibers, polymeric fibers of materials such as polycarbonate, polyethylene, polypropylene, polystyrene, polyethylene terephthalate, polyamide, polyurethane, polyurea, polysulfone, polyvinyl chloride, acrylic and methacrylic polymers, expanded polytetrafluoroethylene (Goretex®), ethylene tetrafluoroethylene, graphite, etc. Polyester mesh made of Dacron® (commercially available from E. I. du Pont de Nemours and Company) or nylon are especially suitable. These materials can be used either alone, or in a composite form in combination with elastomers or hydrogels. Especially advantageous are mesh, woven, non-woven, perforated, or porous formats of these materials which will allow solid anchoring in the implant body.

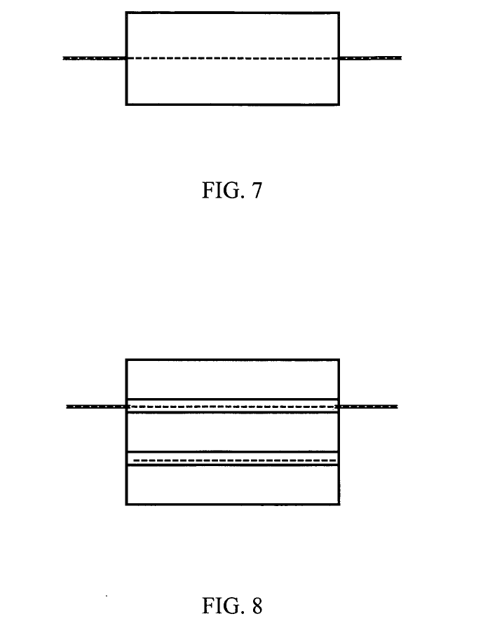

In one embodiment, the implant body may consist of a single polymeric layer in which a support member is embedded. See, e.g., FIG. 7. Alternatively, the support layer may be embedded by being sandwiched between two polymeric layers of the same or differing composition. The polymer can anchor the support member by occupying and surrounding the interstices of a fabric support member and/or by use of an adhesive such as a cyanoacrylate which bonds the support member and the polymer. In a preferred embodiment, at full operational size, the SNI may be composed of at least two substantially parallel soft layers of an elastically deformable polymer such as a hydrogel and at least one relatively rigid layer interposed therebetween, the rigid layer having less compressibility than the soft layers, being adjacent to the soft layers, substantially parallel to them, and firmly attached to them. In some embodiments, the soft layers have the same thickness and/or composition. In other embodiments, the soft layers may have different thickness and/or composition. The implant body may have more than one rigid layer. The rigid layers may have the same or different thickness and/or composition. In one embodiment, the number of soft layers is one more than the number of rigid layers, with, e.g., at least three soft layers. See, e.g., FIG. 8. A support member is preferably embedded in at least one of the relatively rigid layers. It is contemplated that a rigid layer may itself be composed of at least two rigid layers to form a composite rigid layer. A support member can be embedded within or between two of the rigid layers to form a composite rigid layer. As used herein, “rigid layer” or “rigid reinforcing layer” are intended to encompass a single rigid layer and composite rigid layers. As used herein, “full operational size” means the intended final dimensional configuration assumed by the SNI when implanted in a disc space.

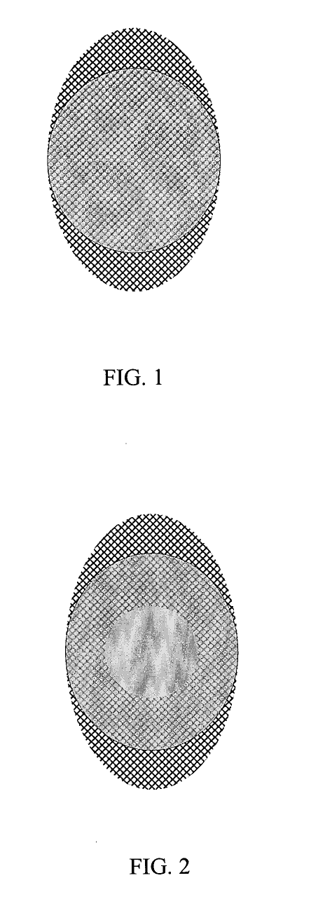

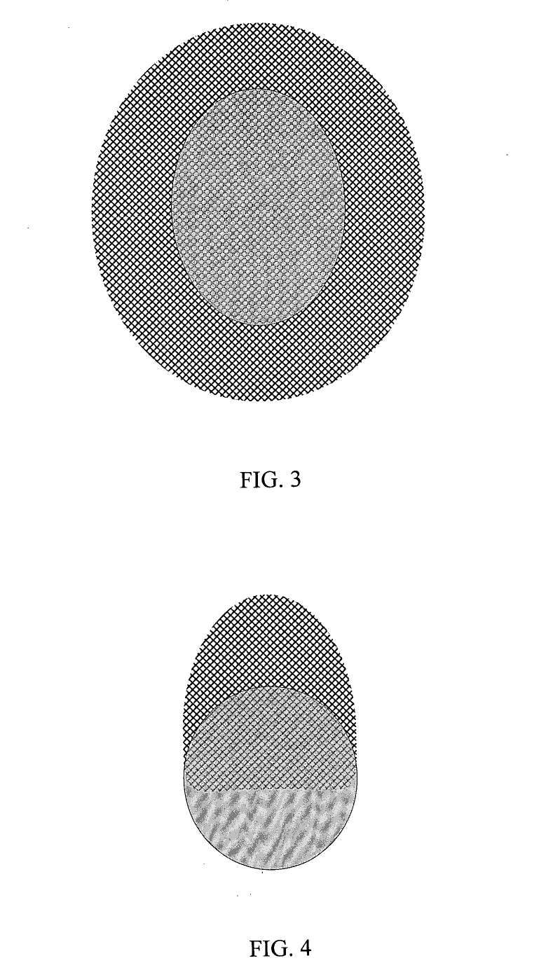

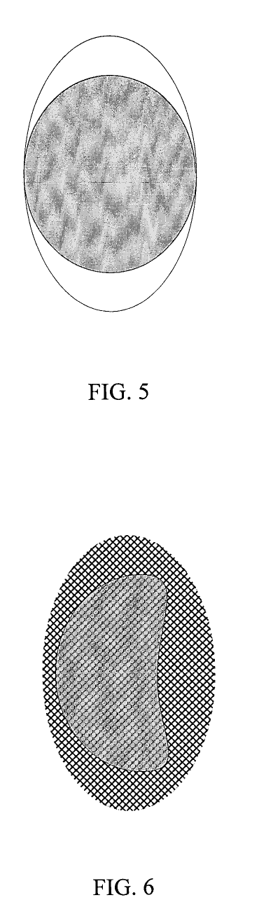

In a preferred embodiment, the implant body is made of hydrogel and is disc-shaped, i.e., cylindrical with a generally ellipsoid footprint when hydrated. The support member may also have a configuration which generally corresponds to the shape of the SNI body footprint when the implant body is at operational size, e.g., the support member having a flat substantially ellipsoid configuration when the implant body has a substantially ellipsoid footprint. See, e.g., FIG. 3. As used herein, “substantially” is intended to mean any of “approximately”, “nearly” or “precisely.” It is also contemplated that the support member may have a shape which is independent of the implant body footprint. Examples of different configurations are shown in FIGS. 1 through 8. FIG. 1 is a top view of a SNI 10. A relatively circular elliptical implant body 12 overlays a more elliptical mesh support member 14. The support member 14 spans the entire ellipsoidal footprint area of the implant body 12 and extends past the implant body 12 at two opposing ends of the ellipsoid. It is preferred that the support member 14 span the entire interior of the implant body 12 to allow a maximum area of adhesion. A support member can, however, be configured to span less than the entire interior of the implant body. See, e.g., FIGS. 2 and 4. FIG. 2 is a top view of a SNI 10′ in which a relatively circular elliptical implant body 12 overlays a more elliptical mesh support member 14′. In this instance, the support member 14′ does not span the entire ellipsoidal footprint area of the implant body 12, i.e., an aperture in the central portion of the support member 14′ is empty. The support member 14′ extends past the perimeter of the implant body 12 at opposite ends. FIG. 4 is a top view of another SNI embodiment 30 in which a circular elliptical implant body 32 overlays a portion of a semi-elliptical support member 34. The support member 34 extends past only one portion of the implant body 32. FIG. 5 is a top view of a SNI 40 in which a relatively circular elliptical implant body 12 overlays an elliptical foil support member 42. The support member 42 spans the entire ellipsoidal footprint area of the implant body 12 and extends past the implant body 12 at two opposing ends of the ellipsoid. In certain embodiments, a support member extends radially beyond the entire perimeter of the implant body. See, e.g., FIGS. 3 and 6. FIG. 3 is a top view of a SNI 20 in which an elliptical implant body 22 overlays a correspondingly shaped mesh support member 24. The support member 24 extends radially beyond the entire perimeter of the implant body 22. FIG. 6 is a top view of a SNI 50 in which a kidney-shaped ellipsoidal implant body 52 overlays an elliptical ellipsoidal mesh support member 14. The support member 14 spans the entire ellipsoidal footprint area of the implant body 52 and extends past the entire perimeter on the body 52. In other embodiments, a support member extends beyond one or more defined portions of the perimeter of the implant body. See FIGS. 1, 2, 4 and 5. Regardless of whether the support member extends past defined portions of the implant body, or the entire perimeter, such extension preferably extends beyond the implant body in substantially parallel orientation relative to the implant body. See, e.g., FIGS. 7 and 8. FIG. 7 is a side view of a single layer SNI 10 having an interiorly embedded support member 14 which extends beyond the periphery of an implant body 12. FIG. 8 is a side view of a five-layer SNI 100. Three softer layers 102 alternate between two more rigid reinforcing layers 104 and 104′ which contain interiorly embedded support members. One support member 104′ is completely contained within the implant body while the other support member 104 extends beyond the perimeter of the implant body. The amount that the support member extends past the implant body in any of the embodiments described herein may vary based on the intended use of the externally disposed portion of the support member. In one embodiment, the perimeter portion of the support member contains barbs for engaging and anchoring to annulus fibers. The barbs may be incorporated at the ends of fibers which make up the mesh, woven, or nonwoven fabric support member. Methods of providing barbed fibers are well-known in the art. For example, barbs may be cast, or physically rendered by blades. Alternatively, barbs may be etched into the body of the fibers using well-known laser techniques.

The implant body may be formed of any biocompatible elastomeric material, i.e., capable of plastic deformation without fracture. Examples include, but are not limited to, natural rubber, silicone, polychloroprene, fluropolymers such as Viton®, ethylene propylene diene monomer (EPDM) rubber, polyurethane, polystyrene, polyvinyl chloride and the like. Hydrogels are especially advantageous for use in forming an implant body herein. Many hydrogel polymers can be deformed, frozen into a deformed shape and can maintain that shape indefinitely or until, e.g., a temperature change causes the polymer to “relax” into the shape originally held prior to freezing. This property is often referred to as shape memory or frozen deformation by those skilled in the art.

The temperature at which frozen deformation occurs is referred to as the glass transition temperature or T_{g}. At T_{g} several polymer properties such as density, entropy and elasticity may sharply change. Many polymers can be mixed with agents that can have a drastic effect on a polymer T_{g}. Polymers which absorb fluid are of particular interest and water is the preferred T_{g} altering agent. Hydrogels which contain less than about five percent water may be considered dehydrated or xerogels. The T_{g} of a xerogel will change as it absorbs fluids containing water. Once the T_{g} becomes lower than ambient, the now partially hydrated hydrogel becomes pliant and may be elastically deformed. If the polymer is held in a state of elastic deformation while the T_{g} is raised above ambient the polymer will maintain the deformed state indefinitely. This can be accomplished by either lowering the ambient temperature (freezing) or by returning the polymer to its xerogel state thus raising the T_{g}.

Using this method, hydrogel articles may be produced with vastly differing xerogel shapes compared to hydrated shapes. This is especially useful in cases such as medical implants where, in delivering a prosthesis into the human body, every care should be taken to reduce trauma to the patient. An implant which is shaped as a cylindrical disc having an ellipsoidal footprint, for instance, may re-shaped, into a tapered elongate rod in order to facilitate minimally invasive implantation. In a preferred embodiment, the support member is flexible, but relatively inelastic, which allows the support member to be bent or folded when the implant body is dehydrated and/or shaped to a compact configuration. An advantage of relative inelasticity is that the support member will not stretch to any large degree, thereby assisting in maintaining the radial dimension of the implant body under load conditions. Once the implant is indwelling and has absorbed water containing liquids it will substantially return to the shape of the cylindrical ellipsoidal disc and maintain that shape indefinitely. As used herein, “disc” is intended to include a round, flattened structure of cylindrical dimension.

Suitable polymers for use in fabricating an implant body herein may contain one or more polymeric components. Preferably, such polymers are made of polymeric components having a C—C backbone. Suitable polymers, such as polyvinylalcohol, polyvinyl pyrrolidone or derivatives of polyacrylic or polymethacrylic acid, are more resistant to biodegradation than polymers with heteroatoms in their backbones, such as polyurethanes or polyesters. Preferably, at least one of the polymeric components contains both hydrophilic and hydrophobic groups.

A preferred polymer configuration includes two polymer phases of different hydrophilicity, the less hydrophilic phase having higher content of hydrophobic groups and more hydrophilic phase having higher content of hydrophilic groups. The less hydrophilic phase is preferably crystalline and more hydrophilic phase is preferably amorphous, as can be established from X-ray diffraction.

Advantageous hydrophobic groups are pendant nitrile substituents in 1,3 positions on a polymethylene backbone, such as poly(acrylonitrile) or poly(methacrylonitrile). The hydrophilic phase may preferably contain a high concentration of ionic groups. Preferred hydrophilic groups are derivatives of acrylic acid and/or methacrylic acid including salts, acrylamidine, N-substituted acrylamidine, acrylamide and N-substituted acryl amide, as well as various combinations thereof. A particularly preferred combination contains approximately two thirds acrylic acid and its salts (on molar basis), the rest being a combination of plain and N-substituted acrylamides and acrylamidines.

At least one polymeric component is preferably a multiblock copolymer with alternating sequences of hydrophilic and hydrophobic groups. Such sequences are usually capable of separating into two polymer phases and form strong physically crosslinked hydrogels. Such multiblock copolymers can be, for example, products of hydrolysis or aminolysis of polyacrylonitrile or polymethacrylonitrile and copolymers thereof. For convenience, polymers and copolymers having at least about 80 molar % of acrylonitrile and/or methacrylonitrile units in their composition may be referred to as “PAN”. Hydrolysis and aminolysis of PAN and products thereof are described, for example, in U.S. Pat. Nos. 4,107,121; 4,331,783; 4,337,327; 4,369,294; 4,370,451; 4,379,874; 4,420,589; 4,943,618, and 5,252,692, each being incorporated herein by reference in their respective entireties.

The SNI can include at least two polymeric components arranged as an interpenetrating network. In that case, one component is essentially a hydrophobic polymer capable of forming a reticulated crystalline fibrillar mesh or scaffold. Examples of such polymers are polyurethane, polyurea, PAN, expanded polytetrafluoroethylene, cellulose triacetate and polyvinylalcohol. The spaces between the fibrils may be filled by a continuous phase of hydrophilic polymer with a 3-dimensional physical or covalent network (i.e., a hydrogel such as crosslinked polyvinylalcohol or polyvinylpyrrolidone). The most suitable hydrogels for this role are those based on hydrophilic derivatives of polyacrylic and polymethacrylic acid.

A preferred material for the SNI is a synthetic composite of a cellular (or domain) type with continuous phase formed by a hydrophobic polymer or a hydrophilic polymer with low to medium water content forming a “closed cell” spongy structure that provides a composite with good strength and shape stability. Examples of suitable polymers are polyurethanes, polyureas, PAN, polydimethylsiloxanes (silicone rubber), and highly crystalline multiblock acrylic and methacrylic copolymers. The polymer should be sufficiently permeable to water. It is known that even distinctly hydrophobic polymers, such as silicone rubber, can form swellable composites. More preferably, the continuous phase is formed by a strong hydrophilic polymer with sufficient permeability for water but impermeable to high-molecular solutes. Examples of such polymers are highly crystalline hydrogels based on segmented polyurethanes, polyvinylalcohol or multiblock acrylonitrile copolymers with derivatives of acrylic acid. Typically, suitable polymers for the continuous phase in cellular composites have a water content in fully hydrated state between about 60% by weight and about 90% by weight, preferably between about 70% and about 85% by weight.

The second component may be a highly hydrophilic polymer of high enough molecular weight to prevent permeation of the hydrophilic polymer through the continuous phase. This component is contained inside the matrix of the continuous phase. The entrapped hydrophilic polymers (the so-called “soft block”) may be high-molecular weight water-soluble polymers, associative water-soluble polymers or highly swellable hydrogels containing, in fully hydrated state, at least about 95% of water and up to about 99.8% of water. Such hydrogels are very weak mechanically. However, it does not matter in composites where such polymers' role is generation of osmotic pressure rather than load-bearing, with compression strength in full hydration in the range of about 0.01 MN/m² or lower.

A system with closed cells (or domains) containing highly swellable or water-soluble polymers can form composites with very high swelling pressure as needed for the SNI function. Examples of suitable hydrophilic polymers are high-molecular weight polyacrylamide, polyacrylic acid, polyvinylpyrrolidone, polyethyleneoxide, copolymers of ethylene oxide and propylene oxide, or hyaluronic acid; covalently crosslinked hydrogels such as hydrophilic esters or amides of polyacrylic or polymethacrylic acids; and physically crosslinked hydrogels, such as hydrolyzates or arninolyzates of PAN.

Particularly suitable are associative water-soluble polymers capable of forming very highly viscous solutions or even soft physical gels. Preferred are associative polymers containing negatively charged groups, such as carboxylates, sulpho-groups, phosphate groups or sulfate groups. Particularly preferred are associative polymers formed by hydrolysis and/or aminolysis of PAN to high but finite conversions that leave a certain number of nitrile groups (typically, between about 5 and 25 molar %) unreacted.

Preferred composites have both a continuous phase and a dispersed phase formed by different products of hydrolysis or aminolysis of PAN. In this case, both components are compatible and their hydrophobic blocks can participate in the same crystalline domains. This improves anchorage of the more hydrophilic component and prevents its extraction or disassociation. The size of more hydrophilic domains may vary widely, from nanometers to millimeters, preferably from tens of nanometers to microns.

The ratio between the continuous discrete phase (i.e., between more hydrophobic and more hydrophilic components may vary from about 1:2 to about 1:100 on a dry weight basis, and a preferred ratio ranges from about 1:5 to about 1:20. Examples of compositions and implants are described in U.S. Pat. Nos. 6,264,695 and 6,726,721, both of which are incorporated herein by reference in their entireties. A preferred method of making the composite is described in U.S. Pat. No. 6,232,406, herein incorporated by reference in its entirety.

Methods of manufacturing SNIs are disclosed, e.g., in U.S. Pat. Nos. 6,264,695 and 6,726,721. Examples of particularly suitable hydrogel forming copolymers are prepared by a partial alkaline hydrolysis of polyacrylonitrile (“HPAN”) in the presence of sodium thiocyanate (NaSCN). The resulting hydrolysis product is a multi-block acrylic copolymer, containing alternating hydrophilic and hydrophobic blocks. Hydrophilic blocks contain acrylic acid, acrylamidine, and acrylamide. In one embodiment, for example, a PAN hydrolysate polymer (referred to herein as HPAN I) (46±1% conversion of hydrolysis) having the following composition: acrylonitrile units ˜53-55%, acrylic acid units ˜22-24%, acrylamide units ˜17-19%, acrylamidine units ˜4-6%, as determined by ¹³C NMR, is dissolved in a suitable solvent such as a ˜55% solution of sodium thiocyanate in water to form a viscous solution. The viscous solution is poured into a porous mold having, e.g., a ring or cylindrical shape. The solution can then be solvent cast, e.g., by solvent exchange (e.g., water for NaSCN). The pores should be sufficiently small as to not permit the polymer to diffuse or leak out of the mold. If desired, a support member, as described herein may be positioned within the mold such that a portion of the support member extends radially out of the mold and liquid polymer is added to fill the mold and surround the portion of the support member that is contained within the confines of the mold. In one embodiment, the mold includes a first ellipsoid ring for receiving liquid polymer and a second ellipsoid ring which fits over the first ellipsoid ring. The first ring is filled with liquid polymer, a support member is placed between the two rings such that a desired portion of the support member extends beyond the perimeter of the ring; the second ring is placed over the first ring in a fluid-tight manner, and liquid polymer is added to fill the second ring. The liquid polymer is then coagulated, e.g., by solvent exchange, and a coagulated implant having a portion of the support member exteriorly disposed is removed from the mold to produce an SNI having an interiorly embedded support member which extends out of the implant body.

If a multilayer implant having alternating softer and stiffer layers is desired, e.g., a more rigid layer, which preferably contains an interiorly embedded support member may then be placed on top of the viscous HPAN I solution which may or may not contain a support member. The more rigid layer may be a preformed hydrogel layer made as described above but, e.g., from another PAN hydrolyzate polymer, referred to herein as HPAN II (28±1% conversion of hydrolysis), having the following composition: acrylonitrile units ˜71-73%, acrylic acid units ˜13-15%, acrylamide units ˜10-12%, acrylamidine units ˜2-4%, as determined by ¹³C NMR, disolved in ˜55% NaSCN which was solvent cast, washed, dried and cut to a suitable shape for fitting over the viscous HPAN I solution in the mold. In certain embodiments, the HPAN II layer may include a support member as described hereinabove which was included during solvent casting. In other embodiments, the support member may be placed over the viscous HPAN I solution in the mold prior to placing the preformed more rigid layer in the mold. Alternatively, the support member may be included in the HPAN I layer(s). HPAN I layers are more hydrophilic than HPAN II layers, are more swellable and have a lower modulus of elasticity.

In one embodiment, a more rigid layer made from, e.g., HPAN II, and containing an embedded support member is optionally dried and placed over a first ellipsoid ring mold filled with HPAN I viscous solution such that at least a portion of the support member extends beyond the perimeter of the mold. A second ellipsoid ring which fits over the first ellipsoid ring in a substantially fluid tight arrangement is placed coaxially over the rigid layer such that at least a portion of the support member extends beyond the perimeter of the mold. The second ring is filled with HPAN I viscous solution. If desired, another preformed, optionally dried hydrogel layer, with or without a support member, is placed over the viscous solution, followed by a third ellipsoid ring mold in fluid-tight arrangement coaxial with the first and second ellipsoid rings. The third ring is filled with viscous HPAN I polymer solution. The process may be repeated until any desired number of layers is formed. The order of layering may be varied to suit particular applications. After the last layer is applied, the mold is closed and placed in water for solvent exchange. For example, the sodium thiocyanate solution diffuses out and is replaced with water, causing the viscous solution to coagulate. In the case of successive layers of HPAN I and HPAN II, the layers adhere to each other without the need for any adhesives. In certain embodiments, the interface between the HPAN I layers and the HPAN II layers is blurred by comingling of the polymers during the manufacturing process, leading to a gradual transition from layer to layer. In other embodiments, the layers may be separately cast and adhesives such as polyurethanes or cyanoacrylates may be used to bond the layers together.

Upon completion of the solvent exchange extraction process SNI are hydrated to their fullest extent (˜90% equilibrium water content (EWC)). In this fully hydrated state the SNI is readily deformed under modest loads and the hydrogel, e.g., HPAN I or HPAN II, glass transition temperature (T_{g}) is well below room temperature. This is the “relaxed” state of the SNI, the state to which it will return after loading below the critical level. The critical level is the point at which permanent deformation occurs and is further discussed below. The fully hydrated SNI is preferably deformed into a desirable second shape and the temperature of the SNI is lowered below its T_{g} (near freezing point of water). Such an SNI would be said to be in a state of “frozen deformation” and it would retain that deformed shape indefinitely. Once the SNI is warmed above its T_{g}, however, the SNI would recover to its original memorized configuration. The support members are advantageously flexible and are free to be bent or folded when compressed during dehydration.

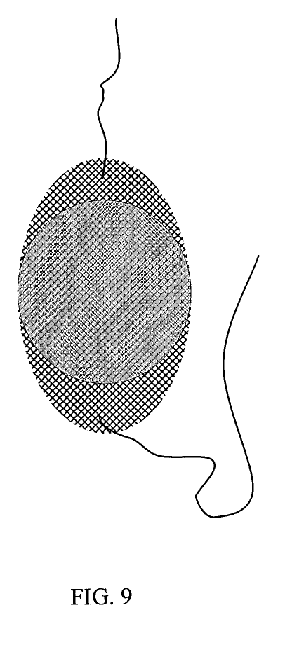

As mentioned above, the amount the support member extends past the implant body may be varied depending on the end use contemplated. By extending the dimensions of the support member beyond the perimeter of the implant body, various modalities for guiding the implant to a desired position in the disc space are provided. In addition, various modalities for anchoring the SNI in the disc space are available. A flexible guide member may be attached to an internal or external portion of the support member which provides a practioner with the ability to manipulate the position of the SNI during and after insertion into the disc space. The guide member may be a string, preferably a suture (mono or multifilament) made from any known suture manufacturing material, a wire (metal or polymeric) or a ribbon (metal or polymeric). The guide member may be permanently or releasably affixed to a support member of an SNI at an interior location proximate to where the support member extends out of the implant body or at any point on the exterior portion of the support member. Multiple guide members may be affixed at different points on the support member. FIG. 9 is a top view of a SNI 10 having an implant body 12, a support member 14 which spans the entire interior of the body 12 and which has two external portions extending from opposite points of the body 12. Two guide members 16 and 16′ are affixed respectively to each of the external portions. The guide member(s) should be long enough to extend from the SNI and out of the disc space to a point where the practioner can comfortably grasp the guide member. It is contemplated that guide members can have varying degrees of flexibility. A slightly flexible, but relatively stiff guide member can be used to both push and pull a SNI in the disc space.

The guide member may be made radiopaque by incorporating a radiopaque material in the guide member. In this manner, the guide member may be visualized using radiographic techniques. For example, a thin radiopaque wire may be wrapped or braided around or within the guide member. Alternatively, radiopaque particles such as metal flakes or grains may be incorporated in a polymeric matrix which forms the guide member. It is contemplated that any technique known to those with skill in the art can be utilized to render the guide member at least partially radiopaque.

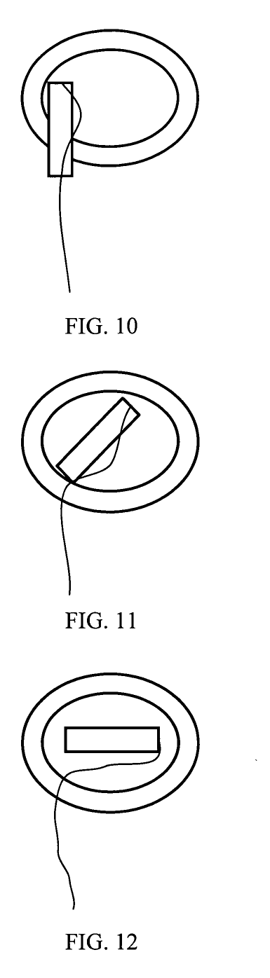

The guide member(s) is especially useful in implantation procedures where a relatively small incision is made in the annulus and a dehydrated rod-shaped implant is inserted through the incision. The techniques described below may be used in both anterior and posterior approaches to SNI implantation. Certain techniques are schematically illustrated in FIGS. 10 through 19. A SNI 200 is inserted through an incision in the annulus 202 into the disc space 204. The SNI 200 is partially inserted and guide member 206 is seen to be trailing the SNI 200 in FIG. 10. In FIG. 11 the SNI 200 is completely inside the disc space 204 and the trailing end has been pushed toward a lateral side of the disc space 204. The guide member 206 is pulled to leverage the leading end of the SNI 200 to cant about 45° relative to its orientation upon insertion. As can be seen from FIG. 12, the leading end has been manipulated via the guide member 206 to cant along an approximately 45° to 100° arc relative to the trailing end.

A typical surgical procedure begins with the patient being placed in a prone position on a lumbar frame. Prior to incision, radiographic equipment can assist in locating the precise intraoperative position of the proposed implantation. Following incision, the facets, lamina and other anatomical landmarks are identified. The affected vertebrae may be distracted using a lamina spreader or a lateral distractor, both of which are commonly known in the art. Following distraction, a transforaminal channel is created by removing the inferior facet of the cranial vertebrae and the superior facet of the caudal vertebrae. A discectomy is performed during which disc material from the affected disc space may be removed using conventional techniques. A SNI 200 is then introduced into the intervertebral disc space 204 via the transforaminal channel and an incision in the annulus 202. The implant 200 is guided along an arcuate path by the guide member 206 to its final position. Once the implant 200 is in the desired final position, such as the symmetric final position shown in FIG. 12, the guide member is optionally removed. If the guide member is made of resorbable polymers such as lactide/glycolide or caprolactone polymers, the guide member 206 may be left in the disc space to be resorbed. In another embodiment, at least a portion of the guide member 206 is cut within the disc space and removed. After implantation, the SNI proceeds to hydrate and swell in the disc space until, in a preferred embodiment, it substantially fills the disc space and provides balanced support to the spinal column. In certain embodiments herein, a first transforaminal channel is created which is configured to receive a spinal nucleus implant and provide relatively good access to one-half the disc space. A second, contra-lateral transforaminal channel, which may have a smaller diameter than the first channel, is created for accessing the other half of the disc space. Discectomy is performed by accessing both respective halves through the closest respective channels. The two-channel approach also allows manipulation of the SNI through both channels.

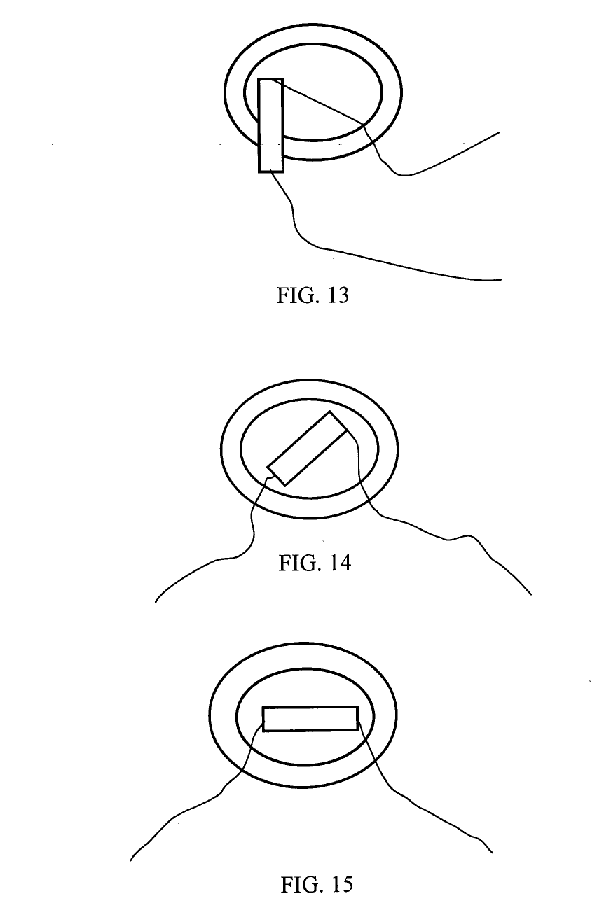

In another embodiment, advantageously suited for a posterior interlaminar approach to SNI implantation, and illustrated schematically in FIGS. 13 through 15, a SNI 300 has two opposing flexible guide members 306 and 308. As shown in FIG. 13, the SNI 300 is partially inserted through a first incision in the annulus 302 into the disc space 304. A second incision is or was made contra-laterally in the annulus and the guide 308 from the leading end of the SNI 300 is grasped by a conventional surgical grasping instrument (not shown) such as forceps, hemostat, snare or a hook and pulled through the second incision. The guide member 306 is affixed to the trailing end of the SNI 300. In FIG. 14 the SNI 300 is completely inside the disc space 304 and the trailing end has been pushed toward a lateral side of the disc space 304 by manipulation of the flexible guide members 306 and 308. The guide member 308 is pulled to leverage the leading end of the SNI 300 to cant about 45° relative to its orientation upon insertion. Flexible guide member 306 is used to stabilize the SNI 300 as guide 308 is pulled. As can be seen from FIG. 15, the leading end of the SNI 300 has been manipulated via the guide members 306 and 308 to cant along an approximately 45° to 100° arc relative to the trailing end. In one embodiment, either, or both, of the guide members are stiff enough to allow them to be used as pushing instruments against the implant.

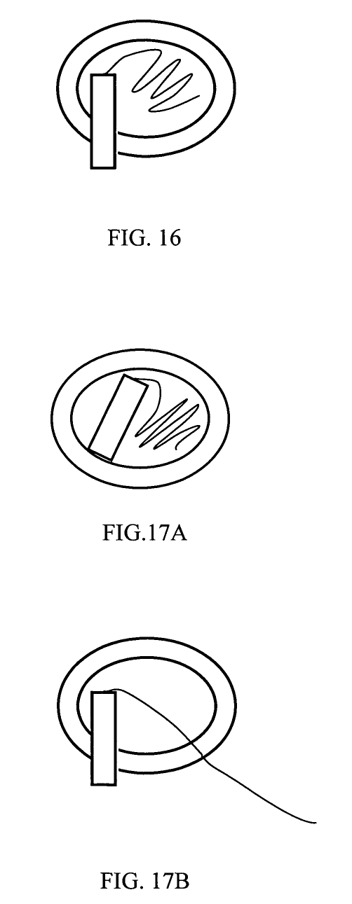

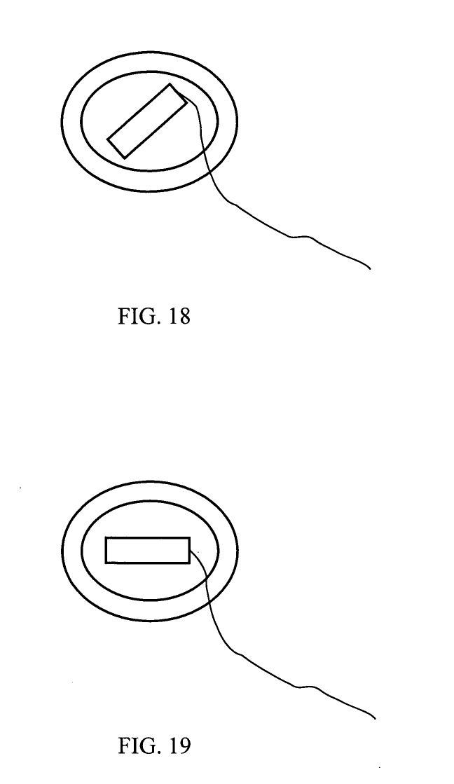

In another embodiment, advantageously suited for a posterior interlaminar approach to SNI implantation, and illustrated schematically in FIGS. 16 through 19, the SNI 200 is inserted such that guide member 206 is completely inserted into the disc space 404. As shown in FIG. 16, the SNI 200 is partially inserted through a first incision in the annulus 402 into the disc space 404. The SNI 200 may be fully inserted as shown in FIG. 17A such that both the SNI 200 and the guide member 206 are contained in the disc space. A second incision is or was made contra-laterally in the annulus and the guide member 206 is grasped by a conventional surgical grasping instrument (not shown) such as forceps, hemostat or a hook and pulled through the second incision. See FIG. 18. The guide member 206 is pulled to leverage the leading end of the SNI 200 to cant about 45° relative to its orientation upon insertion. The trailing end of the SNI 200 may be pushed further into the disc space through the first incision while the guide member 206 is manipulated to cause the leading end of the SNI 200 to cant along an approximately 45° to 100° arc relative to the trailing end. See FIG. 19. In one embodiment, the guide member 206 is stiff enough to allow it to be used as a pushing instrument against the implant. In an alternative embodiment, shown in FIG. 17B, the guide member 206 is pulled through the second incision before the SNI 200 is fully inserted into the disc space 404. After the guide member 206 has been secured outside the disc space 404, the SNI 200 is then pushed completely into the disc space 404 as shown in FIG. 18. The guide member 206 is then optionally removed by cutting or by any other suitable means. It should be understood that although the schematic illustrations of FIGS. 10-19 appear to show the respective guide members attached to the implant body, it is contemplated that the guide member(s) can advantageously be attached to the support member at one or more positions.

After a SNI has been implanted in the disc space, additional extrusion resistance and implant stability may be obtained by attachment of the SNI to the annulus or vertebral bone by sutures, staples, screws, clips or other fasteners. Such attachment may be difficult in the case of viscoelastic implants, especially high water content hydrogels where rigid materials can easily tear out at high stress point, e.g., a point of attachment for a suture or other fastener. A support member as described herein provides ideal points of attachment for fasteners, especially in the externally disposed areas. For example, fasteners such as screws and the like may be used to fasten the support member to a vertebral end plate. The support member distributes the stress of the attachment throughout its own surface area which is well bonded to the SNI. A fabric or foil support member may be stapled, sewn, screwed or otherwise fastened to the annulus or bone, thereby stabilizing the SNI within the disc space. It is contemplated that the support member may optionally be made of a heavier, more durable material when utilized to receive such sutures, screws, clips or other fasteners to prevent the support member from ripping or degrading at the point or points of attachment. Alternatively, or in conjunction with heavier, more durable material, further reinforced areas of the support member may be incorporated to support the point or points of contact between, e.g., a screw, the support member and annulus or bone. Further reinforcement may be accomplished by, e.g., increasing denier of the support member or by adhering a reinforcement element such as a pledget or an additional swatch of support member to or over the portion of the support member at such points of contact. Grommets may be employed to further decrease stress at the point or points of contact between the fastener and the support member. Those skilled in the art may use any conventional method for attaching the reinforcement element to the support member. The guide members may be utilized for attaching the SNI in the disc space, e.g., by using them as sutures and suturing to the annulus. Accordingly, the exteriorly disposed portion of the support member should, e.g., extend from the implant body in an amount ranging from about 1 mm to about 50 mm or more. As mentioned above, the perimeter portion of the support member may also contain barbs for engaging the annulus. The barbs may be used alone or in combination with other fasteners to reduce the possibility of extrusion.

In one embodiment, the support member is used to anchor a suture, e.g., a guide member as described above, which is used to close the annulus after insertion of the implant. In this manner, the guide member can actually serve three purposes, namely, 1) help guide the implant into and in the disc space, 2) anchor the implant in the disc space by virtue of its attachment to the annulus, and 3) a closure mechanism for the incision in the annulus. The free end of the guide member may be fitted with a suture needle which is then used to suture the annulus closed. After tying off the suture, the needle is removed. In another embodiment, the support member is used to patch the annulus at the incision or any suspected weak points. Accordingly, a portion of the support member extending beyond the periphery of the implant body is adapted and configured to be folded or otherwise manipulated to abut the annulus and cover the incision or other target area like a blanket. A suture may then be used to sew the support member to the annulus, thus sealing the incision and/or securing the support member to the annulus. The suture may be initially unattached to the support member or it could be pre-attached to the support member as described above and used as a guide member prior to suturing.

In addition, the exteriorly disposed portion of the fabric support member serves as an ideal medium for ingrowth of connective tissue within the disc space which serves to anchor the SNI within the disc space. For example, Type I collagen is known to proliferate within a damaged disc space and provides an ideal modality for ingrowth into the interstices of the support member, especially in the case of a mesh. In one embodiment, medicinal agents such as connective tissue growth enhancement agents are coated or otherwise imbedded in the exteriorly disposed portion(s) of the support member. Growth factors such as insulin-like growth factors, transforming growth factor β, and connective tissue growth factor, morphogenic proteins, antimicrobials, anti-inflammatory agents may be utilized to promote connective tissue ingowth. The length of the exteriorly disposed portion of the support member may vary from about 5 mm to about 50 mm or more for this purpose. It is contemplated that the exterior portion may be long enough to cover the implant body when folded over.

It should be understood that the examples and embodiments provided herein are preferred embodiments. Various modifications may be made to these examples and embodiments without departing from the spirit and scope of the accompanying claims. For example, those skilled in the art may envision additional polymers, materials and/or hydrogels not mentioned herein which can be utilized herein for the implant body, the support member and the guide member. Similarly, the shapes of the hydrated SNIs and support members described herein are exemplary and any suitable hydrated or dehydrated SNI shape or support member shape can be utilized. Multiple, complementary SNI bodies may be utilized to fill the disc space. Although the interiorly embedded support member is preferably disposed within the implant body in substantially parallel orientation to the implant body footprint, it may be oriented at many different angles including perpendicular to the footprint. In addition, process parameters such as temperature, humidity, pressure, time and concentration may be varied according to conventional techniques by those skilled in the art to optimize results.

What is claimed is:

1. A spinal nucleus implant comprising an implant body and an interiorly embedded support member which extends out from the implant body, said implant adapted and configured to fit within an intervertebral disc space.

2. A spinal nucleus implant according to claim 1, wherein the body has an ellipsoid footprint.

3. A spinal nucleus implant according to claim 1, wherein the interiorly embedded support member is disposed within the implant body in substantially parallel orientation to the footprint.

4. A spinal nucleus implant according to claim 3, wherein the interiorly embedded support member extends beyond the body substantially parallel to the footprint.

5. A spinal nucleus implant according to claim 1, wherein, the support member extends radially beyond and around the entire periphery of the body.

6. A spinal nucleus implant according to claim 1, wherein the support member extends beyond at least one defined portion of the periphery of the body.

7. A spinal nucleus implant according to claim 1, wherein the support member is configured to extend and be folded over a portion of the surface area of the body.

8. A spinal nucleus implant according to claim 7, wherein the support member is configured to extend and be folded over a majority of the surface area if the body.

9. A spinal nucleus implant according to claim 1, wherein the support member is fabric selected from the group consisting of mesh, woven fabric and nonwoven fabric.

10. A spinal nucleus implant according to claim 1, wherein the fabric is made from a material selected from the group consisting of natural polymers, synthetic polymers and metal fibers.

11. A spinal nucleus implant according to claim 1, wherein the support member is a foil made from metal or a polymer.

12. A spinal nucleus implant according to claim 1, wherein the body is made of at least two layers and the support member located between two layers.

13. A spinal nucleus implant according to claim 1, wherein the body is made of alternating substantially parallel layers wherein at least one of the layers contains the support member.

14. A spinal nucleus implant according to claim 1, wherein the support member is at least partially encapsulated by a polymeric coating.

15. A spinal nucleus implant according to claim 1, wherein the support member includes an uncoated portion which is located outside of the body, said portion adapted to engage a guide for orienting the implant.

16. A spinal nucleus implant according to claim 15, wherein the guide is selected from the group consisting of wire, ribbon or string.

17. A spinal nucleus implant according to claim 15, wherein the guide is releasably affixed to the support member.

18. A spinal nucleus implant according to claim 1, wherein the support member is adapted to promote ingrowth of tissue.

19. A spinal nucleus implant according to claim 18, wherein the support member incorporates a medicinal agent which promotes tissue growth.

20. A spinal nucleus implant according to claim 1, wherein the body is made of an elastomeric material.

21. A spinal nucleus implant according to claim 20, wherein the elastomeric material is selected from the group consisting of natural rubber, vulcanized rubber, silicone, polychloroprene, fluropolymers, ethylene propylene diene monomer (EPDM) rubber, polyurethane, polyurea, polystyrene, and polyvinyl chloride.

22. A spinal nucleus implant according to claim 20, wherein the elastomeric material is a hydrogel.

23. A spinal nucleus implant according to claim 22, wherein the hydrogel is selected from the group consisting of polyacrylonitrile, polyvinylalcohol, polyvinylpyrrolidone and derivatives of polyacrylic or polymethacrylic acid.

24. A spinal nucleus implant according to claim 1, wherein the implant is capable of expanding from a compact, substantially dehydrated configuration to an expanded hydrated configuration.

25. A spinal nucleus implant comprising an implant body and an elongate flexible guide member attached to the implant.

26. A spinal nucleus implant according to claim 25 wherein the guide member is selected from the group consisting of wire, ribbon and string.

27. A spinal nucleus implant according to claim 26 wherein the string is a suture.

28. A spinal nucleus implant according to claim 27 wherein the suture is resorbable.

29. A spinal nucleus implant according to claim 25 wherein the guide member is affixed to a support member which is embedded in the interior of the implant body.

30. A spinal nucleus implant according to claim 29 wherein the guide member is releasably affixed to the support member.

31. A spinal nucleus implant according to claim 29 wherein the support member is fabric selected from the group consisting of mesh, woven fabric and nonwoven fabric.

32. A spinal nucleus implant according to claim 29 wherein the support member is a foil made from metal or a polymer.

33. A spinal nucleus implant according to claim 29, wherein the support member is adapted to promote ingrowth of tissue.

34. A spinal nucleus implant according to claim 33, wherein the support member incorporates a medicinal agent which promotes tissue growth.

35. A spinal nucleus implant according to claim 25, wherein the body is made of an elastomeric material.

36. A spinal nucleus implant according to claim 35, wherein the elastomeric material is selected from the group consisting of natural rubber, vulcanized rubber, silicone, polychloroprene, fluropolymers, ethylene propylene diene monomer (EPDM) rubber, polyurethane, polyurea, polystyrene, and polyvinyl chloride.

37. A spinal nucleus implant according to claim 35, wherein the elastomeric material is a hydrogel.

38. A spinal nucleus implant according to claim 37, wherein the hydrogel is selected from the group consisting of polyacrylonitrile, polyvinylalcohol, polyvinylpyrrolidone and derivatives of polyacrylic or polymethacrylic acid.

39. A spinal nucleus implant according to claim 25, wherein the body incorporates layers, wherein certain layers have a different modulus of elasticity compared to other layers.

40. A spinal nucleus implant according to claim 39, wherein the layers are a series of layers which alternate between one having a higher modulus of elasticity and one having a lower modulus of elasticity.

41. A spinal nucleus implant according to claim 40, wherein at least one layer having a higher modulus of elasticity contains the support member at least partially embedded therein.

42. A spinal nucleus implant according to claim 39, wherein at least one of the layers includes a support member having a polymeric coating.

43. A spinal nucleus implant according to claim 25, wherein the implant is capable of expanding from a compact, substantially dehydrated configuration to an expanded hydrated configuration.

44. A spinal nucleus implant according to claim 25, wherein the guide member is at least partially radiopaque.

45. A method of manufacturing a spinal nucleus implant comprising:
providing a liquid polymer;
providing a mold for containing the polymer;
providing a support member;
positioning the support member relative to said mold such that liquid polymer can at least partially cover the support member; and
coagulating the liquid polymer such that at least a portion of said support member extends beyond the perimeter of the polymer to form a spinal nucleus implant having an interiorly disposed support member which extends out of the polymer.

46. A method of manufacturing a spinal nucleus implant according to claim 45 wherein the mold includes a first ellipsoid ring portion for receiving liquid polymer and a second ellipsoid ring portion for disposing over the first ellipsoid ring portion and receiving liquid polymer, wherein positioning the support member relative to the mold involves:
filling the first ring with said liquid polymer;
placing the support member over the first ring such that at least a portion of said support member extends beyond the perimeter of the first ring;
positioning the second ring coaxially over the first ring and the support member to produce a substantially liquid-tight arrangement between the first and second rings;
filling the second ring with liquid polymer; and
coagulating the liquid polymer to form the spinal nucleus implant having an interiorly disposed support member which extends out of the polymer.

47. A method of manufacturing a spinal nucleus implant according to claim 46 further comprising:
providing a first additional ellipsoid ring mold;
filling the first additional mold with liquid polymer;
placing the implant having an interiorly disposed support member coaxially over the first additional ellipsoid ring mold and in contact with the liquid polymer; and
coagulating the liquid polymer such that the polymer adheres to the implant having an interiorly disposed support member as it coagulates to form a spinal nucleus implant having a first polymeric layer containing the support member and a second polymeric layer, wherein the support member extends beyond the perimeter of the polymeric layers.

48. A method of manufacturing a spinal nucleus implant according to claim 47, wherein the first polymer layer containing the support member has a different modulus of elasticity than the second polymeric layer.

49. A method of manufacturing a spinal nucleus implant according to claim 46 further comprising:
providing a second additional ellipsoid ring mold;
placing said second additional mold coaxially over the first polymer layer containing the support member;
filling the mold with liquid polymer; and
coagulating the liquid polymer such that the polymer adheres to the first polymer layer containing the support member as it coagulates to form a three polymeric layer spinal nucleus implant wherein the support member extends beyond the perimeter of at least one of the polymeric layers.

50. A method of manufacturing a spinal nucleus implant according to claim 49 further comprising:
providing a second polymeric layer containing a support member;
placing the second polymeric layer containing the support member coaxially over the second ellipsoid ring mold and in contact with the liquid polymer contained by the second ellipsoid ring mold; and
coagulating the liquid polymer such that the polymer adheres to the second polymeric layer containing the support member as it coagulates to form a four polymeric layer spinal nucleus implant.

51. A method of manufacturing a spinal nucleus implant according to claim 50 wherein the support layer extends beyond the perimeter of at least one of the polymeric layers.

52. A method of manufacturing a spinal nucleus implant according to claim 51 further comprising:
providing a third additional ellipsoid ring mold;
placing said third additional mold coaxially over the second polymeric layer containing the support member;
filling the third additional ellipsoid ring mold with liquid polymer; and
coagulating the liquid polymer such that the polymer adheres to the second polymeric layer containing the support member as it coagulates to form a five polymeric layer spinal nucleus implant.

53. A method of manufacturing a spinal nucleus implant according to claim 46 wherein the modulus of elasticity of the coagulated polymer of the polymeric layer having an interiorly disposed support members is greater than the modulus of elasticity of the layer which does not have an interiorly disposed support member.

54. A method of manufacturing a spinal nucleus implant according to claim 45, wherein the polymer is an elastomeric material.

55. A method of manufacturing a spinal nucleus implant according to claim 54, wherein the elastomeric material is selected from the group consisting of natural rubber, vulcanized rubber, silicone, polychloroprene, fluropolymers, ethylene propylene diene monomer (EPDM) rubber, polyurethane, polyurea, polystyrene, and polyvinyl chloride.

56. A method of manufacturing a spinal nucleus implant according to claim 54 wherein the elastomeric material is a hydrogel.

57. A method of manufacturing a spinal nucleus implant according to claim 56, wherein the hydrogel is selected from the group consisting of polyacrylonitrile, polyvinylalcohol, polyvinylpyrrolidone and derivatives of polyacrylic or polymethacrylic acid.

58. A method of manufacturing a spinal nucleus implant according to claim 45, wherein the support member is a fabric selected from the group consisting of woven fabric, nonwoven fabric and mesh.

59. A method of manufacturing a spinal nucleus implant according to claim 45, wherein the support member is a foil made from metal or a polymer.

60. A method of implanting a spinal nucleus implant comprising:
providing a spinal nucleus implant having a proximal portion and a distal portion, the distal portion having an elongated flexible guide member affixed thereto, the guide member having a proximal end and a distal end, the proximal end being affixed to the distal portion of the implant;
providing a point of entry to the disc space between two vertebrae;
inserting the implant into the disc space using the distal portion of the implant as the leading portion of the implant through the point of entry; and
manipulating the guide member to cause the implant to change position.

61. A method of implanting a spinal nucleus implant according to claim 60 wherein said change in position involves canting in arcuate fashion.

62. A method of implanting a spinal nucleus implant according to claim 61 wherein canting in arcuate fashion encompasses an arc ranging from approximately ˜45° to ˜100° relative to the proximal portion.

63. A method of implanting a spinal nucleus implant according to claim 60 wherein the guide member is selected from the group consisting of string, wire and ribbon.

64. A method of implanting a spinal nucleus implant according to claim 63 wherein the string is a suture.

65. A method of implanting a spinal nucleus implant according to claim 64 wherein the suture is resorbable.

66. A method of implanting a spinal nucleus implant according to claim 60 wherein the guide member is affixed to an interiorly embedded support member which extends out from the implant body, the guide member being affixed to a portion of the support member which extends out from the implant body.

67. A method of implanting a spinal nucleus implant according to claim 62 wherein the distal end of the guide remains outside the point of entry and manipulating the guide includes pulling on the guide member to pull the distal portion of the implant along the arc.

68. A method of implanting a spinal nucleus implant according to claim 60 further comprising:
providing a second point of entry into the disc space, using a grasping instrument to grasp the guide member from within the disc space, and using the grasping instrument to pull on the guide member and cause the implant to change position.

69. A method of implanting a spinal nucleus implant according to claim 68 wherein the grasping instrument is selected from the group consisting of forceps, hemostat and hook.

70. A method of implanting a spinal nucleus implant according to claim 68 wherein the proximal portion of the spinal implant has a second guide member attached thereto.

71. A method of implanting a spinal nucleus implant according to claim 70 further comprising using the second guide member to manipulate the position of the spinal nucleus implant.

72. A method of implanting a spinal nucleus implant comprising inserting, through an entry point of an annulus, a spinal nucleus implant comprising an implant body and an interiorly embedded support member which extends out from the implant body, said implant adapted and configured to fit within an intervertebral disc space.

73. A method of implanting a spinal nucleus implant according to claim 72 wherein the support member extends beyond at least one defined portion of the periphery of the body.

74. A method of implanting a spinal nucleus implant according to claim 73 further comprising positioning the support member against the annulus to cover the entry point and fastening the support member to the annulus.

75. A method of implanting a spinal nucleus implant according to claim 74 wherein the fastening is accomplished using a fastener selected from the group consisting of suture, staple, screw and clip.

76. A method of implanting a spinal nucleus implant according to claim 75 wherein the fastener is a suture which is attached to the support member.

77. A method of implanting a spinal nucleus implant according to claim 73 wherein the support member has a suture attached to it.

78. A method of implanting a spinal nucleus implant according to claim 77 wherein the suture is used to guide the implant into the intervetebral disc space.

79. A method of implanting a spinal nucleus implant according to claim 77 further comprising suturing and closing the entry point with the suture after implantation of the implant.

80. A method of implanting a spinal nucleus implant according to claim 73 further comprising fastening the support member to vertebral bone.

81. A method of implanting a spinal nucleus implant according to claim 80 wherein fastening is accomplished using a fastener selected from the group consisting of screw, staple, and barb.

82. A method of implanting a spinal nucleus implant according to claim 80 wherein the vertebral bone is a vertebral end plate.

83. A method of implanting a spinal nucleus implant according to claim 73 wherein the support member includes a reinforced area for contacting a fastener.
